(19) United States
(12) Patent Application Publication
(10) Pub. No.: US 2023/0166063 A1
(43) Pub. Date: Jun. 1, 2023

(54) VENTILATOR AND INFLATION BAG OPERATION OF VENTILATOR

(71) Applicant: Kuwait University, Safat (KW)

(72) Inventor: Bader Shafaqa Al-Anzi, Al Sabah (KW)

(73) Assignee: Kuwait University, Safat (KW)

(21) Appl. No.: 17/536,370

(22) Filed: Nov. 29, 2021

(51) Int. Cl. A61M 16/00; A61M 16/20

(52) U.S. Cl. CPC A61M 16/0078; A61M 16/208; A61M 2205/078; A61M 2205/3379

(57) ABSTRACT

A ventilator includes at-least one air vessel, at-least one inflation bag, at-least one connecting member, a bag holder and at-least one controller member. The at-least one connecting member connects to the at-least one air vessel. The bag holder may hold the inflation bag. The at-least one controller member operates the at-least one inflation bag thereby changing size of the inflation bag to vary a tidal volume and an inspiration-expiration ratio of the ventilator. In another aspect, at-least one controller member is mounted on the bag holder. Further, the at-least one controller member slides within the bag holder to change the shape of the inflation bag to vary a tidal volume and an inspiration-expiration ratio of the ventilator.

### TECHNICAL FIELD

The embodiments herein are generally related to the field of medical ventilator. The embodiments herein are particularly related to a ventilator, a controller member and an inflation bag. The embodiments herein are more particularly related to a ventilator and a controller member for operating an inflation bag of a ventilator.

### BACKGROUND

The ongoing time is the time of transmissible diseases outbreak which resulted in pandemic conditions. The pandemic condition causes significant disturbance in economic, social, and political harmony. Statistics related to past pandemics suggest that the likelihood of pandemic conditions may be intensified due to globalization or higher global travel and integration. Multiple outbreaks, notable ones are COVID-19, West Africa Ebola epidemic in 2014, a severe acute respiratory syndrome (SARS) pandemic in 2003, had shown cracks (e.g., early detection of disease, availability of basic medical facility, contacts of infected person, quarantine and isolation procedures etc.) during pandemic circumstances. These unprecedented pandemic circumstances had significantly impacted humanity, claiming many innocent lives and costing billions of dollars. Significant attention has been paid by the international community for development of strategy to alleviate and/rheostat pandemics.

One such strategy to alleviate the challenges of pandemic conditions and similar future problems is to provide sufficient medical ventilators to the humans who got infected by viruses during these unprecedented pandemic times. A conventional medical ventilator needs to precisely deliver an air-oxygen mixture at prescribed oxygen concentrations to the distressed patient's lungs, and the delivery should be carefully made at predefined/prescribed volumes, delivery pressures, and delivery rates. The conventional medical ventilators are built with valves, array of sensors, oxygen concentrators, power backup systems, multi-mode operations, data acquisition, alarms, sophisticated algorithms, electronic controllers etc. to do a complex array of activities in a very sensitive manner by sensing the patient condition and responding to his needs. These components drive up the final cost of the medical ventilator as well as limit large-scale production of the ventilators at a faster rate.

Intermittent Positive Pressure Ventilation (IPPV) is the widely employed method for artificial respiration for the patient experiencing difficulty in performing natural respiration. Ambu bags, commonly known as bag valve masks or manual resuscitators, are simple resuscitators that are most widely used respiratory support equipment in medical emergency situations. Mostly, when a patient who is not in the vicinity of hospital needs breathing support or cardiopulmonary resuscitation (CPR), the bag-valve-mask (BVM) or the Ambu bag is used. The bag-valve-mask (BVM) or the Ambu bag is a self-inflating bag which releases air from one end port when squeezed and sucks air to refill itself with fresh air from another end port, when released. The volume of air pushed into the lungs of the patient when the airbag is pressed is normally referred to as Tidal Volume. The bag-valve-mask (BVM) or the Ambu bag can be operated by pneumatic control using valves or even by motor. In both, inspiration to expiration duration Ratio (IER) is varied by changing “Pause or Stop” duration. Drive motion is not continuous as there is ‘stop’ or ‘pause’ in each cycle. This mechanism requires a special motor like Servo motor, stepper motor, etc. which can be electronically controlled. This increases complexity and cost of the ventilator.

In addition, in certain situations, doctors or nurses may require more accustomed ventilators, and may look to find portable ventilators, in the manner or requirement they prefer.

The above-mentioned shortcomings, disadvantages, and problems are addressed herein, which will be understood by studying the following specifications.

### SUMMARY

This summary is provided to introduce a selection of concepts in a simplified form that are further described below in the Detailed Description. This summary is not intended to identify key features or essential features of the claimed subject matter, nor is it intended as an aid in determining the scope of the claimed subject matter.

According to one aspect of the present disclosure, a ventilator is disclosed. The ventilator includes at-least one air vessel, at-least one inflation bag, at-least one connecting member, a bag holder and at-least one controller member. The at-least one connecting member to connect the at-least one air vessel with the at-least one inflation bag. The bag holder is configured to hold the inflation bag. The at-least one controller member is operably configured to operate the at-least one inflation bag thereby changing size of the inflation bag to vary a tidal volume and an inspiration-expiration ratio of the ventilator.

According to another aspect of the present disclosure, a ventilator is disclosed. The ventilator includes at-least one air vessel, at-least one inflation bag, at-least one connecting member, a bag holder and at-least one controller member. The at-least one connecting member to connect the at-least one air vessel with the inflation bag. The bag holder is configured to hold the inflation bag. The at-least one controller member is mounted on the bag holder. Further, the at-least one controller member is operably configured to slide within the bag holder to change the shape of the inflation bag to vary a tidal volume and an inspiration-expiration ratio of the ventilator.

According to another aspect of the present disclosure, a ventilator is disclosed. The ventilator includes an air vessel, an inflation bag, a connecting member, a bag holder and a controller member. The connecting member connects the air vessel with the inflation bag. The bag holder is configured to hold the inflation bag. The controller member is mounted on the connecting member. Further, the controller member is operably configured to provide resistance to the pressured air going to the inflation bag from the air vessel thereby varying tidal volume and inspiration-expiration ratio of the ventilator.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

A better understanding of embodiments of the present disclosure (including alternatives and/or variations thereof) may be obtained with reference to the detailed description of the embodiments along with the following drawings, in which:

FIGS. 1 and 2 illustrate a schematic view of a pneumatic ventilator having direct supply of air to an inflation bag of the ventilator, in accordance with an exemplary embodiment of the present disclosure;

FIGS. 3a and 3b illustrate an operation of multiple inflation bag in a bag holder, in accordance with an exemplary embodiment of the present disclosure;

FIGS. 4a and 4b illustrate an operation of an inflation bag by a foot pedal member, in accordance with another exemplary embodiment of the present disclosure;

FIGS. 5a, 5b and 5c illustrate schematic view of multiple pneumatic ventilators having indirect supply of air to the multiple inflation bags of the ventilators, in accordance with another exemplary embodiment of the present disclosure;

FIGS. 6a and 6b illustrate another schematic view of a pneumatic ventilator having a small disposable air vessel of air to supply air to an inflation bag of the ventilator, in accordance with another exemplary embodiment of the present disclosure; and

Like reference numerals refer to like parts throughout the description of several views of the drawing.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having an ordinary skill in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfil the requirements of uniqueness, utility and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do NOT necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should NOT be necessarily taken as limiting factors to the attached claims. The attached claims and their legal equivalents can be realized in the context of embodiments other than the ones used as illustrative examples in the description below.

Embodiments of the present invention will be described below in detail with reference to the accompanying drawings.

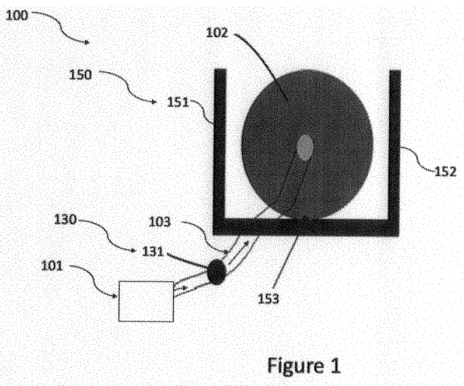

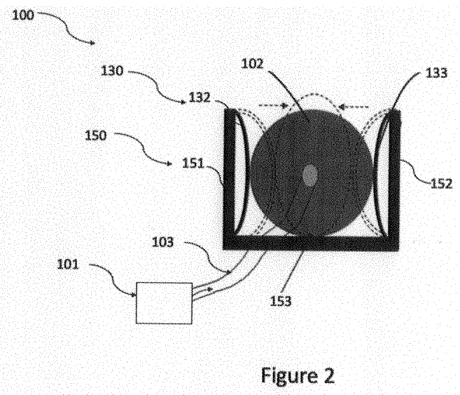

FIGS. 1 and 2 illustrate a schematic view of a pneumatic ventilator having direct supply of air to an inflation bag of the ventilator, according to an embodiment of the present disclosure. In an embodiment, the ventilator 100 is disclosed. The ventilator 100 may include, but is not limited to, at-least one air vessel 101, an inflation bag 102, a connecting member 103, a bag holder 150 and a controller member 130. The air vessel 101 may be a compressor for providing pressured air to the inflation bag 102. The inflation bag 102 may be a bag-valve-mask (BVM) or AMBU bag. The bag-valve-mask (BVM) is adapted to be squeezed and expanded. The connecting member 103 is to connect the air vessel 101 with the inflation bag 102. The bag holder 150 is configured to hold the inflation bag 102. The bag holder 150 comprises a left wall 151, a right wall 152, and a bottom wall 153. The bottom wall 153 connects the left wall 151 with the right wall 152. The controller member 130 is changing the size of the inflation bag 102 by doing contraction and expansion of the inflation bag 102. The controller member 130 contracts the inflation bag 102 to push fresh air into the patient's lungs, and when at-least one controller member 130 comes backward, the at-least one controller member 130 expands the inflation bag 102 and air comes out of the patient's lungs.

Referring to FIG. 1, the controller member 130 comprises a regulator member 131. The regulator member 131 of the controller member 130 is mounted on the connecting member 103. Further, the regulator member 131 is operably configured to provide resistance to the pressured air going to the inflation bag 102 from the air vessel 101 thereby varying tidal volume and inspiration-expiration ratio of the ventilator 100. As the regulator member 131 is regulating air flow inside the inflation bag 102 by regulating the air flow from the air vessel 101 to the inflation bag 102, the air is supplied from the inflation bag 102 to the patient's lungs as per his requirement of air. Further, in another embodiment, the controller member may be an arm or a weight member or a spring-operated member to regulate size of the inflation bag by providing resistance to the pressured air going to the inflation bag 102 from the air vessel 101, without departing from the scope of the present disclosure.

Referring to FIG. 2, the at-least one controller member 130 comprises a left controller member 132 and a right controller member 133. The left controller member 132 and the right controller member 133 are operably configured to operate the inflation bag 102 thereby changing size of the inflation bag 102 to vary a tidal volume and an inspiration-expiration ratio (IER) of the ventilator 100. The left controller member 132 is mounted to the left wall 151 of the bag holder 150. The right controller member 133 is mounted to the right wall 152 the bag holder 150. The left controller member 132 and the right controller member 133 may be sliding over the bottom wall 153 of the bag holder 150. Firstly, the inflation bag 102 receives compressed air from the air vessel 101 and the inflation bag 102 expands. Secondly, the left controller member 132 and the right controller member 133 may expand and slide over the bottom wall 153 of the bag holder 150 for contracting the inflation bag 102. Therefore, the left controller member 132 and the right controller member 133 may change size of the inflation bag 102 by doing contraction and expansion of the inflation bag 102. Finally, the air is supplied from the inflation bag 102 to the patient's lungs as per his requirement of fresh air. As the left controller member 132 and the right controller member 133 contracts the inflation bag 102 to push fresh air in to patient's lungs, and when the left controller member 132 and the right controller member 133 comes backward, the left controller member 132 and the right controller member 133 gives space to the inflation bag 102 for expansion and air comes out of the patient's lungs.

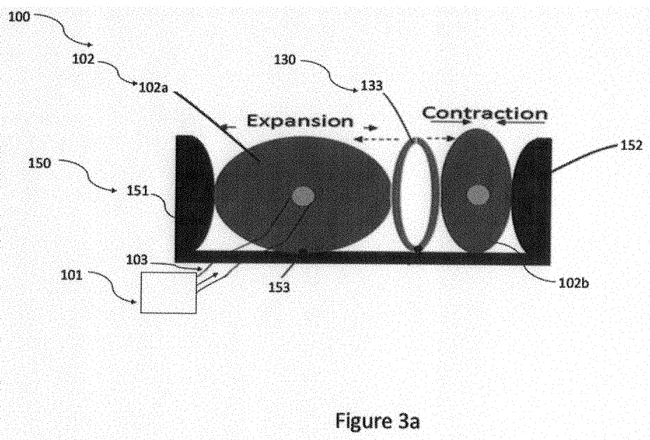

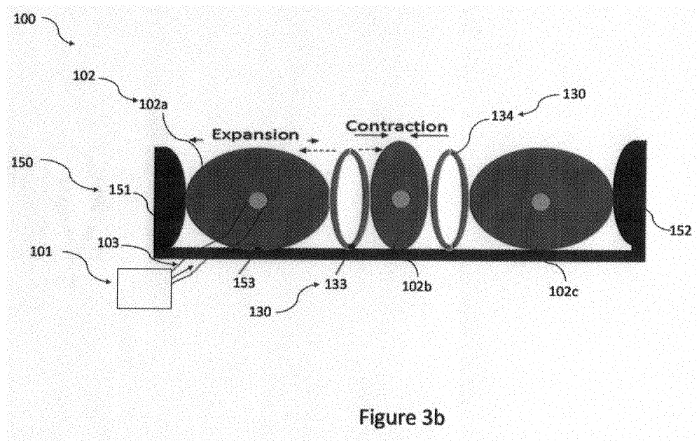

FIGS. 3a and 3b illustrate an operation of the multiple inflation bag 102a, 102b, 102c in the bag holder 150, according to an embodiment of the present disclosure. The ventilator 100 may include, but is not limited to, the at-least one air vessel 101, the at-least one inflation bag 102a, 102b, 102c, the at-least one connecting member 103, the bag holder 150 and the at-least one controller member 130. The at-least one connecting member 103 may be provided to connect the at-least one air vessel 101 with one of the inflation bags out of the at-least one inflation bag 102a, 102b, 102c. The at-least one inflation bag 102 comprises a first inflation bag 102a, a second inflation bag 102b, and a third inflation bag 102c. The bag holder 150 is configured to hold the at-least one inflation bag 102a, 102b, 102c. The at-least one controller member 130 may be mounted on the bag holder 150. In another embodiment, the at-least one controller member 130 may be disposed on the bag holder 150.

Further, the at-least one controller member 130 is operably configured to slide within the bag holder 150 to change the shape of the inflation bag to vary a tidal volume and an inspiration-expiration ratio of the ventilator 100, without departing from the scope of the present disclosure. The at-least one controller member 130 comprises a first controller member 133 and a second controller member 134. For the sake of brevity, details of the present disclosure that are explained in detail in the description of FIG. 1 and FIG. 2 are not explained in detail in the description of FIG. 3a and FIG. 3b.

In an embodiment, at-least one controller member 130 may be sliding over the bottom wall 153 of the bag holder 150. Firstly, the first inflation bag 102a receives compressed air from the air vessel 101 and the first inflation bag 102a expands. Secondly, the first controller member 133 may slide over the bottom wall 153 of the bag holder 150 thereby contracting one of the second inflation bag 102b, and the third inflation bag 102c. As one of the second inflation bag 102b, and the third inflation bag 102c is pressed against one of the right wall 152 of the bag holder 150 and the second controller member 134, at-least one controller member 130 may change size of the inflation bag 102 by doing contraction and expansion of one of the first inflation bag 102a, the second inflation bag 102b and the third inflation bag 102c of the inflation bag 102. Finally, the air is supplied from one of the contracting bags of the inflation bag 102 to the patient's lungs as per his requirement of fresh air. When at-least one controller member 130 causes expansion of one of the expanding bags of the inflation bag 102, the air comes out of the patient's lungs. The present embodiment ensures that the ventilator 100 uses only one controller member 130 or the arm 130 to operate more than one inflation bag 102 or AMBU bags 102 thereby supplying fresh air to a greater number of patients at the same time.

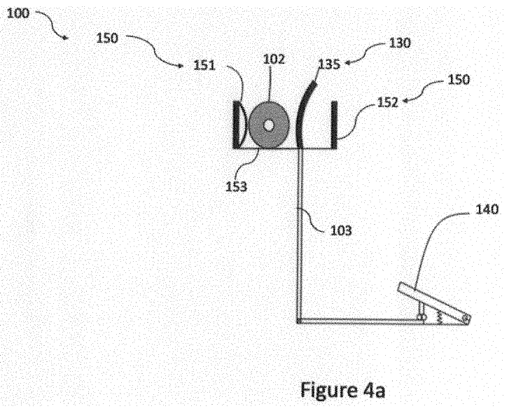

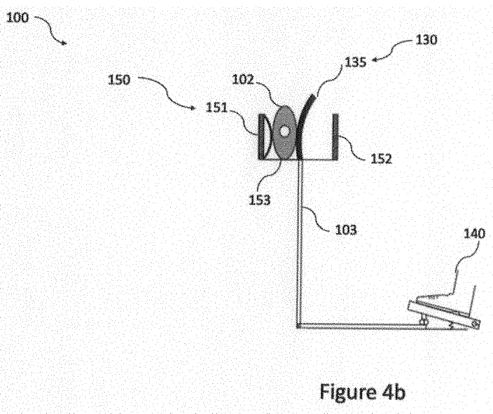

FIGS. 4a and 4b illustrate an operation of the inflation bag 102 by a foot pedal member 140, according to another embodiment of the present disclosure. The controller member 130 comprises a moveable member 135. The foot pedal member 140 is connected to the moveable member 135 through the connecting member 103. In an embodiment, the connecting member 103 may be a cable or rod or metal linkage. However, in another embodiment, the connecting member 103 may be a fluid operated cable also for transferring pressure from the foot pedal member 140 to the moveable member 135. The foot pedal member 140 is operated by a user such as a doctor or nurse.

The moveable member 135 slides over the bottom wall 153 of the bag holder 150 thereby contracting the inflation bag 102. As the inflation bag 102 is pressed against the left wall 152 of the bag holder 150. Therefore, the controller member 130 may change size of the inflation bag 102 by doing contraction and expansion of the inflation bag 102. Hence, the air is supplied from the inflation bag 102 to the patient's lungs as per his requirement of fresh air. Later, the controller member 130 causes expansion of the inflation bag 102, the air comes out of the patient's lungs. The present embodiment ensures that during the usage of the ventilator 100, the user (doctor or nurse) hands remain free thereby giving the user full control to focus on monitoring the patients. For the sake of brevity, details of the present disclosure that are explained in details in the description of FIG. 1, FIG. 2, FIG. 3a and FIG. 3b, are not explained in detail in the description of FIG. 4a, and FIG. 4b.

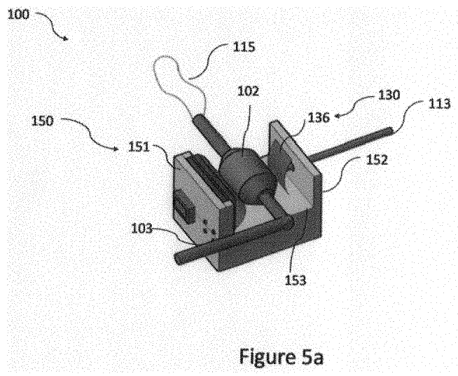

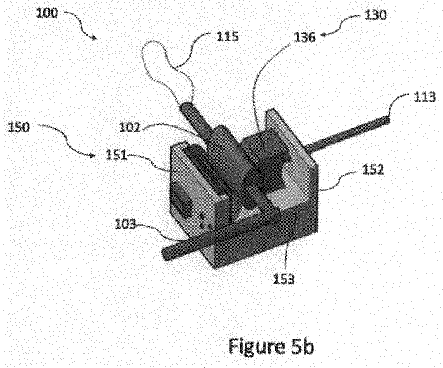

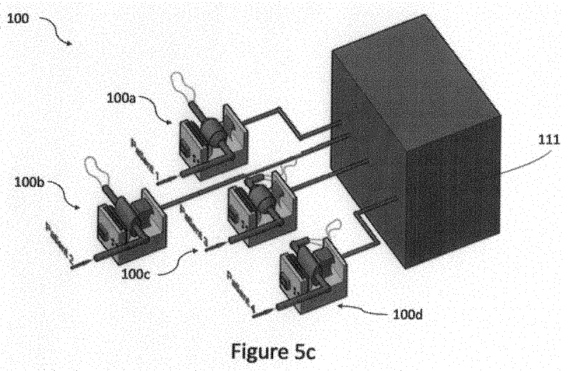

FIGS. 5a and 5b illustrate schematic views of pneumatic ventilators 100 and its operation, according to another embodiment of the present disclosure. Further, FIG. 5c illustrate schematic view of multiple pneumatic ventilators 100a, 100b, 100c, and 100d having indirect supply of air to the multiple inflation bags of the ventilators 100a, 100b, 100c, and 100d, according to another embodiment of the present disclosure. As seen in FIGS. 5a, 5b and 5c, the controller member 130 comprises an inflatable cushion bag 136. The inflatable cushion bag 136 is connected to another air vessel 111 through another connecting member 113. The compressed air flows from another air vessel 111 to the inflatable cushion bag 136 of the controller member 130. As the cushion bag 136 of the controller member 130 is expanded the inflation bag 102 is pressed against the left wall 152 of the bag holder 150, the controller member 130 may change size of the inflation bag 102 by doing contraction and expansion of the inflation bag 102 through the inflatable cushion bag 136. Hence, the air is supplied from the inflation bag 102 to the patient's lungs from an air outlet 115 as per his requirement of fresh air. Later, the controller member 130 causes expansion of the inflation bag 102, the air comes out of the patient's lungs. Therefore, all the multiple pneumatic ventilators 100a, 100b, 100c, and 100d operated in the same manner as described for the ventilator 100. For the sake of brevity, details of the present disclosure that are explained in details in the description of FIG. 1, FIG. 2, FIG. 3a, FIG. 3b, FIG. 4a and FIG. 4b are not explained in detail in the description of FIG. 5a, FIG. 5b and FIG. 5c.

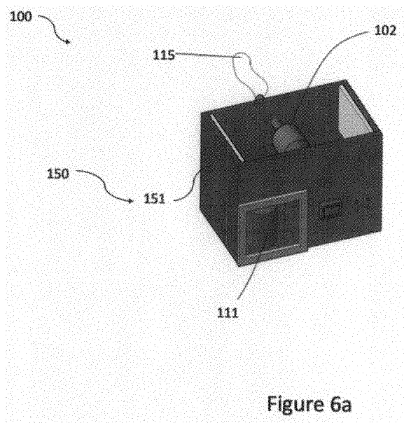

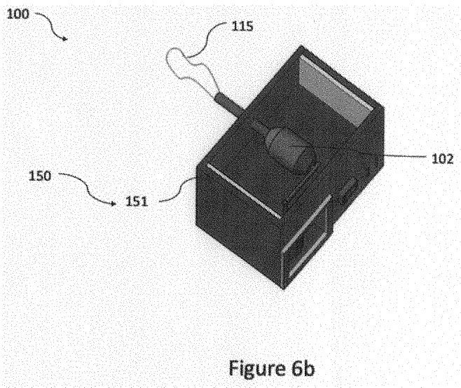

FIGS. 6a and 6b illustrate another schematic view of a pneumatic ventilator having a small disposable air vessel of air to supply air to an inflation bag of the ventilator, according to another embodiment of the present disclosure. The controller member 130 comprises a cushion bag (not shown). The inflatable cushion bag is connected to a small disposable air vessel 111. The compressed air flows from the small disposable air vessel 111 to the cushion bag of the controller member 130. As the controller member 130 is expanded thereby pressing the inflation bag 102 against the bag holder 150. Therefore, the controller member 130 may change size of the inflation bag 102 by doing contraction and expansion of the inflation bag 102. Hence, the air is supplied from the inflation bag 102 to the patient's lungs from the air outlet 115 as per his requirement of fresh air. Later, the controller member 130 causes expansion of the inflation bag 102, the air comes out of the patient's lungs. For the sake of brevity, details of the present disclosure that are explained in details in the description of FIG. 1, FIG. 2, FIG. 3a, FIG. 3b, FIG. 4a, FIG. 4b, FIG. 5a, FIG. 5b and FIG. 5c are not explained in detail in the description of FIG. 6a, and FIG. 6b.

The present disclosure provides a ventilator, such as the ventilator 100. Further, the ventilator 100 of the present disclosure includes at-least one air vessel 101, at-least one inflation bag 102, at-least one connecting member 103, a bag holder 150 and at-least one controller member 130. In particular, the at-least one air vessel 101 may be a compressor 101 for providing pressured air to the inflation bag 102. The at-least one inflation bag 102 is a bag-valve-mask (BVM) or AMBU bag. The bag-valve-mask (BVM) is adapted to be squeezed and expanded. The at-least one connecting member 103 is to connect the air vessel 101 with the inflation bag 102. The bag holder 150 is configured to hold the inflation bag 102. The bag holder 150 comprises a left wall 151, a right wall 152, and a bottom wall 153. The bottom wall 153 connects the left wall 151 with the right wall 152. The at-least one controller member 130 is changing size of the inflation bag 102 by doing contraction and expansion of the inflation bag 102. The at-least one controller member 130 contracts the inflation bag 102 to push fresh air into the patient's lungs, and when the at-least one controller member 130 comes backward, the at-least one controller member 130 expands the inflation bag 102 and air comes out of the patient's lungs.

The present disclosure provides the ventilator 100 that is very simple to use and is cost effective. Also, the present disclosure provides the ventilator 100 that uses only one controller member 130 or the arm 130 to operate more than one inflation bag 102 or AMBU bags 102 thereby supplying fresh air to a greater number of patients at the same time. Moreso, the present disclosure provides the ventilator 100 that may be operated without a power (electricity).

The present disclosure further provides the ventilator 100 which does not require highly skilled professionals for its operation. Also, the present disclosure further provides the ventilator 100 which is portable and lightweight.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope.

Although the embodiments herein are described with various specific embodiments, it will be obvious for a person skilled in the art to practice the embodiments herein with modifications. Although the embodiments herein are described with various specific embodiments, it will be obvious for a person skilled in the art to practice the embodiments herein with modifications.

The scope of the embodiments of the present invention is to be ascertained with the claims to be submitted at the time of filing the complete specification.

What is claimed is:

1. A ventilator comprising:
at-least one air vessel;
at-least one inflation bag;
at-least one connecting member to connect the at-least one air vessel and the at-least one inflation bag; and
at-least one controller member configured to operate the at-least one inflation bag thereby changing size of the inflation bag to vary a tidal volume and an inspiration-expiration ratio of the ventilator.

2. The ventilator of claim 1, wherein the at-least one air vessel is a compressor for providing pressured air to the inflation bag.

3. The ventilator of claim 1, wherein the at-least one controller member is configured to change size of the inflation bag by performing contraction and expansion of the inflation bag.

4. The ventilator of claim 1, wherein the at-least one controller member is at-least one pushing member for performing to and fro motion with the bag holder.

5. The ventilator of claim 1, wherein the inflation bag is a bag-valve-mask and the bag-valve-mask is configured to be squeezed and expanded.

6. The ventilator of claim 1, wherein the at-least one controller member contracts the inflation bag to push fresh air into patient's lungs, and when the at-least one controller member comes backward, the at-least one controller member expands the inflation bag and air comes out of the patient's lungs.

7. A ventilator comprising:
at-least one air vessel;
at-least one inflation bag;
at-least one connecting member to connect the at-least one air vessel and the at-least one inflation bag;
a bag holder configured to hold the inflation bag; and
at-least one controller member mounted on the bag holder, wherein the at-least one controller member is configured to slide within the bag holder to change the shape of the inflation bag to vary a tidal volume and an inspiration-expiration ratio of the ventilator.

8. The ventilator of claim 7, wherein the at-least one controller member is operatively configured with the inflation bag and configured to move in to and fro motion, wherein when the at-least one controller member moved towards the inflation bag, the at-least one controller member presses the inflation bag.

9. The ventilator of claim 7, wherein the at-least one controller member comprises a left controller member and a right controller member, wherein the left controller member and the right controller member operatively configured with the inflation bag and configured to move in to and fro motion.

10. The ventilator of claim 9, wherein the left controller member and the right controller member moved towards the inflation bag, and presses the inflation bag, and wherein when the left controller member and the right controller member comes backward, the left controller member and the right controller member un-presses the inflation bag.

11. The ventilator of claim 9, wherein the left controller member and the right controller member is an expandable cushion member, such that the expansion of the expandable cushion member facilitates compressing of the inflation bag.

12. The ventilator of claim 9, wherein the left controller member and the right controller member change the shape of the inflation bag from spherical to elliptical thereby facilitating compressing of the inflation bag.

13. The ventilator of claim 7, wherein the at-least one controller member is an another inflation bag to regulate size of the inflation bag by sliding within the bag holder, wherein the another inflatable bag used as the movable arm.

14. The ventilator of claim 7, wherein the at-least one controller member is an at-least one moveable arm member to regulate size of the inflation bag by sliding within the bag holder, wherein the at-least one moveable arm member is regulating size of the inflation bag by doing contraction and expansion.

15. A ventilator comprising:
an air vessel;
an inflation bag;
a connecting member to connect the air vessel and the inflation bag;
a bag holder configured to hold the inflation bag; and
a controller member mounted on the connecting member, and the controller member is operably configured to provide resistance to the pressured air going to the inflation bag from the air vessel thereby varying tidal volume and inspiration-expiration ratio of the ventilator.

16. The ventilator of claim 15, wherein the controller member is a regulator to regulate size of the inflation bag by providing resistance to the pressured air going to the inflation bag from the air vessel.

17. The ventilator of claim 15, wherein the controller member is an arm to regulate size of the inflation bag by providing resistance to the pressured air going to the inflation bag from the air vessel.

18. The ventilator of claim 15, wherein the controller member is a weight member to regulate size of the inflation bag by providing resistance to the pressured air going to the inflation bag from the air vessel.

19. The ventilator of claim 15, wherein the controller member is a spring-operated member to regulate size of the inflation bag by providing resistance to the pressured air going to the inflation bag from the air vessel.

20. The ventilator of claim 1, wherein the at-least one moveable member is operably connected to a foot operated member, such that the movement of the foot operated member facilitate to and fro movement of the movement of the at-least one moveable member resulting into the pushing of the inflation bag against one of the another inflation bag and a wall of the bag holder.
